(19) United States
(12) Patent Application Publication
(10) Pub. No.: US 2019/0272892 A1
(43) Pub. Date: Sep. 5, 2019

(54) INTEGRATIVE PANOMIC APPROACH TO PHARMACOGENOMICS SCREENING

(71) Applicant: NantOmics, LLC, Culver City, CA (US)

(72) Inventors: Camille R. Schwartz, Santa Cruz, CA (US); John Little, Culver City, CA (US); Charles Joseph Vaske, Santa Cruz, CA (US); John Zachary Sanborn, Santa Cruz, CA (US)

(21) Appl. No.: 16/420,040

(22) Filed: May 22, 2019

Related U.S. Application Data
(63) Continuation-in-part of application No. 16/003,028, filed on Jun. 7, 2018.
(60) Provisional application No. 62/517,022, filed on Jun. 8, 2017.
(60) Provisional application No. 62/567,719, filed on Oct. 3, 2017.
(60) Provisional application No. 62/676,488, filed on May 25, 2018.
(60) Provisional application No. 62/681,050, filed on Jun. 5, 2018.

(51) Int. Cl. G16B 30/10; G16B 30/20; G16H 20/40

(52) U.S. Cl. CPC G16B 30/10; G16H 20/40; G16B 30/20

(57) ABSTRACT

Complex genotypes, especially multiple single nucleotide variances, that may differentially distributed among alleles can be efficiently mapped in each allele of the gene using next generation sequencing of RNA transcripts from the alleles and the allele fraction information of RNA transcripts. Such reconstructed single nucleotide variances among alleles can be associated with the expected effectiveness of the cancer therapy to update or generate the patient's record or adjust the dose and schedule of the cancer therapy to reduce the undesirable effect of the cancer therapy.

This application is a continuation in part of co-pending U.S. application Ser. No. 16/003,028 filed on Jun. 7, 2018, which claims benefit of priority to U.S. provisional applications with the Ser. No. 62/517,022, filed Jun. 8, 2017, and Ser. No. 62/567,719, filed Oct. 3, 2017. This application also claims the benefit of priority to our co-pending U.S. provisional applications 62/676,488 filed on May 25, 2018, and 62/681,050 filed on Jun. 5, 2018. Each of these applications are incorporated by reference in its entirety herein.

### FIELD OF THE INVENTION

The field of the invention is pharmacogenomics analysis in relation to cancer therapy.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Genetic variations across individual patients often influence the individual patient's response to various pharmacological substances; especially those metabolized by specific metabolic pathways and/or enzymes for providing optimal drug efficacy and reduced toxicity. Several genes that affect cancer drug-related phenotypes (e.g., drug efficacy, drug toxicity, etc.), including thiopurine methyltransferase gene (TPMT), a gene encoding a member of cytochrome P450 mixed-function oxidase system (CYP2D6), and organic anion transporting polypeptide 1B1 (SLCO1B1), and further their single nucleotide variances have been identified, which prompts use of genomics information for tailoring cancer therapy for its maximal and optimal results. For example, treatment with mercaptopurine that is a typical treatment for acute lymphoblastic leukemia, may result in life-threatening toxicity for some patients having variant alleles of TPMT, and it is highly recommended that individual genotyping is performed to identify the existence of such fatal allele to the mercaptopurine treatment prior to the treatment. Yet, one of the major obstacles resides in difficulties in mapping the single nucleotide variances that are allele-specific, which cannot be easily identified by traditional genomic gene sequencing, especially due to the large distance between two single nucleotide variances.

To circumvent such difficulties, efforts have been made to use RNA allele frequencies and DNA copy number variations to identify the allele specific single nucleotide point mutations. For example, Edsgard et al. (Bioinformatics, 32 (19), 2016, 3038-3040) discloses haplotype inference using single-cell RNA-seq data that shows specific pattern of read number distributions. The specific patterns of read number distributions are associated with sequencing data to infer whether the two sequence variants are located in the same allele or not. Similarly, Berger et al (2015, Research in Computational Molecular Biology pp 28-29) discloses that single nucleotide variance in two alleles are often shown different read numbers of RNA transcripts, from which haplotypes can be reconstructed using phasing that HapTree-X framework. Yet, none have provided a thorough and large scale screening of allele specific single nucleotide variance distributions in multiple genes among cancer patients of various types that may affect efficacy and/or toxicity of various cancer drugs.

Thus, even if general methods of phasing single nucleotide variations using allele frequency of RNA transcripts are known, it is largely unexplored how cancer therapy can be identified and modified with mapping of allele-specific single nucleotide variations in specific genes related to drug metabolism. Therefore, there remains a need for improved methods and systems to use omics data for comprehensive characterization of single nucleotide variations in alleles of genes of interest among various types of cancer patients that may affect cancer therapy efficacy and toxicity.

### SUMMARY OF THE INVENTION

The inventive subject matter is directed to various methods use omics data for comprehensive characterization of single nucleotide variations in alleles of genes of interest among various types of cancer patients by analyzing pattern of allele fraction distributions among the RNA transcripts including single nucleotide variations. Thus, one aspect of the inventive subject matter includes method of reducing an adverse effect of a cancer therapy in a patient having a tumor. This method comprises a step of obtaining the patient's transcriptomics data that comprises allele fraction information of first and second loci of an RNA molecule transcribed from a gene having first and second nucleotide variations, respectively. Then, the method continues with a step of using allele fraction information to reconstruct a haplotype of the first and second RNA loci. Preferably, the allele fraction information of the first and second RNA loci is derived from a tumor tissue of the patient. Such reconstructed haplotype can then be associated with an expected effectiveness of the cancer therapy, and used to generate or update the patient's record. In some embodiments, the method may further include a step of adjusting recommended dose and schedule of the cancer therapy based on the expected effectiveness. Preferably, the cancer therapy is identified by a pathway analysis using at least two of genomics, transcriptomics, and proteomics data of the patient.

Most typically, the transcriptomics data can be obtained from RNAseq, and the gene is at least one of CYP3A5, CYP2D6, TPMT, F5, DPYD, G6PD, and NUDT15. While the expected effectiveness of the cancer therapy may vary depending on the type of gene and mutations, such may include drug efficacy, drug toxicity, metabolism rates of a drug, and life expectancy of the patient. Thus, in one embodiment, the gene is CYP2D6 and the expected effectiveness comprises an increased toxicity of the cancer therapy by slow metabolism of the cancer therapy.

Preferably, the first and second RNA loci are at least 300 bp apart, at least 500 bp apart, or at least 1 kbp apart in the RNA transcripts such that the RNA-seq sequence data of the first and second loci do not overlap. The haplotype is reconstructed to have the first and second nucleotide variations in an allele of the gene when the allele fractions of the first and second RNA loci having the first and second nucleotide variations differ less than 10%, less than 15%, or less than 20%.

Additionally, the transcriptomics data may comprise a copy number of the first and second RNA loci, and the method may further comprise a step of determining amplification of at least one of first and second loci of the RNA transcript and generating or updating the patient's record with amplification information of the gene in relation to the expected effectiveness of a cancer therapy. In such embodiment, the gene can be CYP2D6 and the expected effectiveness may comprise a reduced efficacy of the cancer therapy by fast metabolism of the cancer therapy.

Further, the transcriptomics data may also include allele fraction information of the first and second RNA loci derived from a healthy tissue of the patient. In such embodiment, the method may further include steps of using the allele fraction information of the healthy tissue to reconstruct a healthy tissue haplotype, and comparing the allele fraction information derived from the tumor tissue with the allele fraction information derived from the healthy tissue to obtain tumor-specific allele fraction information. Then the patient's record can be generated or updated with the allele fraction information and the tumor-specific allele fraction information. In addition, recommended dose and schedule of the cancer therapy can be further adjusted based on a comparison of the reconstructed healthy tissue's haplotype and the tumor-specific haplotype.

In yet another aspect of the inventive subject matter, the inventors contemplate a method of treating a patient having a tumor. This method comprises a step of obtaining the patient's transcriptomics data that comprises allele fraction information of first and second RNA loci of an RNA molecule transcribed from a gene having first and second nucleotide variations, respectively. Then, the method continues with a step of using allele fraction information to reconstruct a haplotype of the first and second RNA loci. Preferably, the allele fraction information of the first and second RNA loci is derived from a tumor tissue of the patient. An expected effectiveness of the cancer therapy can be inferred for the haplotype and recommended dose and schedule of the cancer therapy can be adjusted or determined based on the inferred expected effectiveness. Preferably, the cancer therapy is identified by a pathway analysis using at least two of genomics, transcriptomics, and proteomics data of the patient.

Most typically, the transcriptomics data can be obtained from RNAseq, and the gene is at least one of CYP3A5, CYP2D6, TPMT, F5, DPYD, G6PD, and NUDT15. While the expected effectiveness of the cancer therapy may vary depending on the type of gene and mutations, such may include drug efficacy, drug toxicity, metabolism rates of a drug, and life expectancy of the patient. Thus, in one embodiment, the gene is CYP2D6 and the expected effectiveness comprises an increased toxicity of the cancer therapy by slow metabolism of the cancer therapy.

Preferably, the first and second RNA loci are at least 300 bp apart, at least 500 bp apart, or at least 1 kbp apart such that the RNA-seq sequence data of the first and second loci do not overlap. The haplotype is reconstructed to have the first and second nucleotide variations in an allele of the gene when the allele fractions of the first and second RNA loci having the first and second nucleotide variations differ less than 10%, less than 15%, or less than 20%.

Additionally, the transcriptomics data may comprise a copy number of the first and second loci of the RNA transcripts, and the method may further comprise a step of determining amplification of at least one of first and second RNA loci and generating or updating the patient's record with amplification information of the gene in relation to the expected effectiveness of a cancer therapy. In such embodiment, the gene can be CYP2D6 and the expected effectiveness may comprise a reduced efficacy of the cancer therapy by fast metabolism of the cancer therapy.

Further, the transcriptomics data may also include allele fraction information of the first and second RNA loci derived from a healthy tissue of the patient. In such embodiment, the method may further include steps of using the healthy tissue allele fraction information to reconstruct a healthy tissue haplotype and comparing the allele fraction information derived from the tumor tissue with the allele fraction information derived from the healthy tissue to obtain tumor-specific allele fraction information. Recommended dose and schedule of the cancer therapy can be then adjusted using the allele fraction information and the tumor-specific allele fraction information. In addition, the patient's record can be further generated and/or updated with the reconstructed haplotype in relation to an expected effectiveness of the cancer therapy.

Various objects, features, aspects and advantages of the inventive subject matter will become more apparent from the following detailed description of preferred embodiments and accompanied drawings.

### BRIEF DESCRIPTION OF THE DRAWING

FIG. 1A depicts an exemplary graph of DNA allele fractions in normal and tumor tissue of a patient.

FIG. 1B depicts an exemplary graph of tumor RNA allele fraction against tumor DNA allele fractions of a patient.

FIG. 2A shows an exemplary graph of tumor RNA allele fraction against normal DNA allele fraction of a patient where two single nucleotide variances (α and β) are in the same haplotype.

FIG. 2B shows an exemplary graph of tumor RNA allele fraction against tumor DNA allele fraction of a patient where two single nucleotide variances (α and β) are in the same haplotype.

FIG. 3A shows a graph of read coverage for each exon of CYP2D6 and CYP2D7 gene without any deletion or amplification of alleles.

FIG. 3B shows a graph of read coverage for each exon of CYP2D6 and CYP2D7 gene with allele deletion.

FIG. 3C shows a graph of read coverage for each exon of CYP2D6 and CYP2D7 gene with allele amplification.

### DETAILED DESCRIPTION

The inventors contemplate that genomic variations among patients, especially in genes related to metabolizing chemical substances in the patient's liver, influence the effectiveness of various cancer treatment including cancer drugs. Such genomic variations are often allele-specific (e.g., present in only one of two alleles) and/or across several exons or introns such that it is difficult to map the allele-specific genomic variations throughout a gene and throughout multiple genes. In addition, while it is often necessary to conduct genomic screening covering multiple genomic variances in multiple genes of patients to optimize the types and treatment regimen of the cancer treatments, a comprehensive packet of large scale genomic variation screenings for different types of cancer patients has been unaccounted for.

Viewed from a different perspective, the inventors discovered that allele specific genomic variations can be readily determined using allele fraction information of RNA molecules whose sequences are overlapped in the area where the genomic variations are present and further reconstructing the haplotype with the allele information. The inventors also found that allele fraction information of RNA molecules can be obtained from a patient for multiple genes that are related to drug efficacy and/or toxicity such that the drug treatment plan can be tailored and customized. Consequently, in one especially preferred aspect of the inventive subject matter, the inventors contemplate a method of reducing an adverse effect of a cancer therapy in a patient having a tumor by reconstructing haplotypes having multiple allele-specific single nucleotide variations in one or more gene using allele fraction information. Such reconstructed haplotype information can be used to generate or update the patient's record in relation to an expected effectiveness of the cancer therapy.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

As used herein, the term “locus” (or in plural, “loci”) refers to a portion of or a location in a gene, a transcript of a gene, or a nucleic acid molecule derived from a gene or a transcript of a gene.

### Obtaining Omics Data

Any suitable methods and/or procedures to obtain omics data are contemplated. For example, the omics data can be obtained by obtaining tissues from an individual and processing the tissue to obtain DNA, RNA, protein, or any other biological substances from the tissue to further analyze relevant information. In another example, the omics data can be obtained directly from a database that stores omics information of an individual.

Where the omics data is obtained from the tissue of an individual, any suitable methods of obtaining a tumor sample (tumor cells or tumor tissue) or healthy tissue from the patient are contemplated. Most typically, a tumor sample or healthy tissue sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, tissues or cells may be fresh or frozen. In other example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a healthy tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or healthy tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

Where it is desired to obtain the tumor-specific omics data, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, omics data analysis may reveal missense and nonsense mutations, changes in copy number, loss of heterozygosity, deletions, insertions, inversions, translocations, changes in microsatellites, etc.

Moreover, it should be noted that some data sets are preferably reflective of a tumor and a matched normal sample of the same patient to so obtain patient and tumor specific information. In such embodiments, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Preferably, the genomics data includes allele-specific sequence information and copy number. In such embodiment, the genomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

In addition, omics data of cancer and/or normal cells comprises transcriptome data set that includes sequence information and expression level (including expression profiling, copy number, or splice variant analysis) of RNA(s) (preferably cellular mRNAs) that is obtained from the patient, from the cancer tissue (diseased tissue) and/or matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

It should be appreciated that one or more desired nucleic acids or genes may be selected for a particular disease (e.g., cancer, etc.), disease stage, specific mutation, or even on the basis of personal mutational profiles or presence of expressed neoepitopes. Alternatively, where discovery or scanning for new mutations or changes in expression of a particular gene is desired, RNAseq is preferred to so cover at least part of a patient transcriptome. Moreover, it should be appreciated that analysis can be performed static or over a time course with repeated sampling to obtain a dynamic picture without the need for biopsy of the tumor or a metastasis.

Further, omics data of cancer and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

### Omics Data Analysis and Selection of Cancer Drug as Treatment

The inventors contemplate that a molecular profile or a molecular signature of the tumor tissue can be determined using omics data, preferably two or more types of omics data. While any types or subtypes of omics data may be used to determine the molecular profile or a molecular signature of the tumor tissue, it is contemplated that the type of omics data preferred may differ based on the type of tumor, based on the desired information (e.g., information on intrinsic drug sensitivity, tumor cell stemness, etc.), and/or the prognosis of the tumor (e.g., metastasized, immune-resistant, etc.). Exemplary subtypes of genomics data that may be relevant to tumor development can include, but not limited to genome amplification (as represented genomic copy number aberrations), somatic mutations (e.g., point mutation (e.g., nonsense mutation, missense mutation, etc.), deletion, insertion, etc.), genomic rearrangements (e.g., intrachromosomal rearrangement, extrachromosomal rearrangement, translocation, etc.), appearance and copy numbers of extrachromosomal genomes (e.g., double minute chromosome, etc.). In addition, genomic data may also include tumor mutation burden that is measured by the number of mutations carried by the tumor cells or appeared in the tumor cell in a predetermined period of time or within a relevant time period.

In addition to the genomics data, one or more subtypes of transcriptomics data can be used to determine the molecular profile or a molecular signature of the tumor tissue. Exemplary transcriptomics data includes, but not limited to, expression levels of a plurality of mRNAs as measured by quantities of the mRNAs, maturation levels of mRNAs (e.g., existence of poly A tail, etc.), and/or splicing variants of the transcripts. The number of genes (at least two, at least five, at least ten, at least fifteen, etc.), types of transcripts or RNAs (mRNA, miRNA, etc.), or the selection of genes to determine the molecular profile or a molecular signature of the tumor tissue may vary based on the type of tumor, based on the desired information (e.g., information on intrinsic drug sensitivity, tumor cell stemness, etc.), and/or the prognosis of the tumor (e.g., metastasized, immune-resistant, etc.). For example, the selection of genes and/or the number of genes to determine molecular signature related to tumor stemness may differ, or minimally overlap with the selection of genes and/or the number of genes to determine molecular signature related to cell sensitivity to a specific chemotherapeutic drug. It is contemplated that the genes to be included in the relevant transcriptomics data set to differentiate the tumor samples (from the matched normal or among the tumor samples having different physiological characteristics) may include any tumor-specific genes, inflammation-related genes, DNA repair-related genes (e.g., Base excision repair, Mismatch repair, Nucleotide excision repair, Homologous recombination, Non-homologous end-joining, etc.), genes associated with sensitivity to DNA damaging agents, DNA replication machinery-related genes. Yet, it is also contemplated that the genes to be included in the relevant transcriptomics data set to differentiate the tumor samples may include genes not associated with a disease (e.g., housekeeping genes), including, but not limited to, those related to transcription factors, RNA splicing, tRNA synthetases, RNA binding protein, ribosomal proteins, or mitochondrial proteins, or noncoding RNA (e.g., microRNA, small interfering RNA, long non-coding RNA (lncRNA), etc.).

Optionally, one or more subtypes of proteomics data can be used to determine the molecular profile or a molecular signature of the tumor tissue. Exemplary proteomics data includes, but not limited to, quantities of one or more proteins or peptides, post-translational modification of one or proteins or peptides (e.g., phosphorylation, glycosylation, forming a dimer, ubiquitination, etc.), and/or subcellular localization of the proteins or peptides.

Without wishing to be bound by any specific theory, the inventors contemplate that the mutational profiles and/or the RNA expression profiles of the tumor tissue, either independently or collectively, affect the intracellular signaling networks, which consequently may change the intrinsic properties of the tumor tissues or cells. Thus, so determined mutational profiles and/or the RNA expression profiles of the tumor tissue can be integrated into a pathway model to generate a modified pathway or the tumor-specific pathway. Most typically, the pathway model comprises a plurality of pathway elements (e.g., proteins) that are connected by one or more regulatory nodes. For example, a pathway model [A] is a factor-graph-based pathway model (e.g., PARADIGM pathway model) that comprises pathway elements A, B, and C connected by a regulatory node I between the elements A and B, and another regulatory node II between the element B and C (A-I-B-II-C). The regulatory node I and II represent any factors other than A or B that may affect the activity of B and C. Thus, the pathway model [A] may be coupled to another pathway model [B] via one of the regulatory nodes I and II. Thus, in some embodiments, the pathway model may include a single pathway (e.g., PKA mediated apoptosis pathway, etc.). Consequently, in some embodiments, the pathway model may be a single degree model that includes one or more signaling pathways that are parallel or substantially independent from each other. In other embodiments, the pathway model may be a multi-degree model that may include a plurality of signaling pathways that are coupled via one or more regulatory nodes (e.g., two degree model having pathways [A] and [B] where pathways [A] and [B] are coupled in a regulatory node of the pathway [A], three degree model having pathways [A], [B], and [C] where the pathways [A] and [B] are coupled in a regulatory node of the pathway [A] and pathways [B] and [C] are coupled in a regulatory node of the pathway [B].

The pathway element activity of each pathway element can be inferred or calculated using the omics data as inputs in the central dogma module (DNA-RNA-protein-protein activity) as described in WO 2014/193982, which is incorporated by reference herein. For example, where the gene encoding protein A carries multiple genomic mutations in the exome, and RNA expression level of the gene increase upon a drug treatment, it can be inferred from such genomics and transcriptomics profile, the quantity of the protein may be increased while the activity of such protein may provide a dominant negative effect in the signaling pathway (where protein A is an element of the signaling pathway) due to missense mutations in the critical post-translational modification residues. Based on such inferred individual pathway element activity, the activity of downstream signaling pathway element can be inferred in the same signaling pathway or another signaling pathway that is connected by a regulatory node.

Consequently, diverse types of omics data can be integrated into a single pathway model to so allow on the basis of measured attributes (e.g., DNA copy number and/or mutations, RNA transcription level, protein quantities and/or activities) calculation of inferred attributes (e.g., DNA copy number and/or mutations, RNA transcription level, protein quantities and/or activities for which no data were obtained from the sample) and also calculation of inferred pathway activities. Advantageously, such calculations can employ the entirety of available omics data, or only use omics data that have significant deviations from corresponding normal values (e.g., due to copy number changes, over- or under-expression, loss of protein activity, etc.). Using such system, it should be appreciated that instead of analyzing only single or multiple markers, cell signaling activities and changes in such signaling pathways can be detected that would otherwise be unnoticed when considering only single or multiple markers in disregard of their function.

Preferably, the pathway models can be pre-trained via a machine learning algorithms (e.g., Linear kernel SVM, First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor) with omics data from the healthy individuals as inputs and corroborative data. In such embodiment, through the machine learning algorithms, each pathway element and the factor to the regulatory node will be provided with weights and directions to determine the activity of the downstream pathway elements. For example, where the pathway elements A and B are connected to regulatory node I, each, or at least one of quantity (e.g., copy number, expression level of RNA) and/or status (e.g., types and locations of mutations, number of phosphorylation for phosphorylated protein, etc.) of pathway element A and/or any factors of regulatory node I (e.g., activity of an enzyme affecting the activity of pathway element A, etc.) are integrated or calculated to infer the activity of pathway element B (e.g., quantity, status of protein B).

Consequently, such trained pathway model can be used as a template to predict how the pathway or pathway elements would be changed in the tumor tissue. For example, omics data obtained from the patient (and preferably compared with the matched normal tissue or healthy tissue from healthy individuals) can be integrated into a factor-graph-based model using PARADIGM (or any suitable pathway models that can be machine-trained and produce reliable output data) to infer or predict which and how pathway elements would be changed due to the tumor-specific omics data changes compared to the compared with the matched normal tissue or healthy tissue from healthy individuals. Thus, suitable pathway models include Gene Set Enrichment Analysis (GSEA, Broad Institute) based models, Signaling Pathway Impact Analysis (SPIA, Bioconductor) based models, and PathOlogist pathway models (NCBI) as well as factor-graph based models, and especially PARADIGM as described in WO2011/139345A2, WO2013/062505A1, and WO2014/059036, all incorporated by reference herein.

Thus, genomic mutation profile, RNA expression profile, and optionally proteomic profiling (either measured from the sample or inferred by pathway analysis) can be further used collectively to identify or predict signaling pathway elements in the relevant signaling pathway that are most significantly changed in the tumor tissue such that the most desirable target for tumor treatment(s) can be selected. Further, the inventors also contemplate that based on such pathway analysis, it can be inferred how the activity of the signaling pathway, overall, or even the signaling networks comprising a plurality of signaling pathways is changed or modified in response to an event (e.g., drug treatment, etc.) to indicate increasing sensitivity or susceptibility to the anti-tumor treatment, developing or acquiring resistance to the anti-tumor treatment, or unresponsiveness to the anti-tumor treatment. Thus, pathway analysis in view of drug selection and treatment may provide guidance in selecting the optimal and personalized treatment regime(s) for treating the tumor.

### Phasing RNA Molecules of Different Loci and Determining Allele Haplotype

Even if a cancer drug that has high likelihood of success in treating the tumor is identified from the pathway analysis using patient's omics data, the cancer drug may not be effectively used to treat the patient's tumor if the cancer drug cannot be metabolized in an efficient manner and/or produce toxicity to the patient's normal tissues or cells due to the patient's specific genetic variance. Several genes and single nucleotide variances on those genes that may affect the effectiveness of some currently available cancer drug have been identified. In some of those genes, the effect of each single nucleotide variance and/or combinations of some of single nucleotide variances and/or the combination of different type of alleles having different combinations of single nucleotide variances may vary with respect to the expected effectiveness and/or toxicity of the cancer drug. For example, various allele types of CYP2D6 having distinct combinations of single nucleotide variances and their function levels (normal function, decreased function, no function, etc.) have been identified. Interestingly, where two types of alleles contain common single nucleotide variances 1662G→C and 4181G→C, the gene product has decreased function where such variances are co-present with another single nucleotide variance 100C→T in the same allele, and the gene product has no function where such variances are co-present with other single nucleotide variances 882G→C and 2851C→T in the same allele.

In addition, based on the combination of allele types to form the diplotype of the gene, overall function of the gene may change, which may be coupled with various clinical implications. Table 1 shows representative examples of various alleles of genes that affect the effectiveness of cancer drugs. For example, if a patient has *10 alelle in his/her CYP2D6 gene, it is likely that Tamoxifen treatment to the patient may not be as effective as other patient as endoxifen concentration is low in the patient and the chance of recurrence of the tumor after Tamoxifen treatment is relatively high.

TABLE 1

| Gene | Alleles | Drugs | Clinical Implications |
|---|---|---|---|
| CYP3A5 | *3, *6, *7 | Tacrolimus | Normal metabolizers may fail to reach |
| | | | target dose |
| TPMT | *2, *3A, *3B, | Azathioprine, | Increased risk of myelosuppression |
| | *3C, *4 | Mercaptopurine, | and potentially fatal toxicities |
| | | Thioguanine | |
| F5 | rs6025 | Eltrombopag Olamine | Increased risk of thromboembolism |
| DPYD | *2A, *3, *4, | Fluorouracil, | Increased risk of severe or life |
| | *5, *6, *7, *8, | Capecitabine, Tegafur | threatening adverse events |
| | *9A, *9B, | | |
| | *10, *11, *12, | | |
| | *13, | | |
| | rs67376798 | | |
| UGT1A1 | *28 | Belinostat, Irinotecan, | Increased risk of toxicities, |
| | | Nilotinib, Pazopanib | neutropenia, hyperbilirubinemia, |
| | | | hyperbilirubinemia (respectively) |
| G6PD | Mediterranean, | Rasburicase, Dabrafenib | Increased risk of hemolytic anemia |
| | A- | | |
| NUDT15 | *3, *4 | Mercaptopurine | Increased risk of myelotoxicity |
| | | | (leukopenia or neutropenia) |
| HLA- | 07:01 | Lapatinib | Increased risk of hepatotoxicity |
| DRB1 | | | |
| HLA- | 02:01 | Lapatinib | Increased risk of hepatotoxicity |
| DQA1 | | | |
| CYP2D6 | *10 | Tamoxifen | Lower endoxifen concentration, |
| | | | increased likelihood of recurrence |

Thus, in one aspect of the inventive subject matter, allele haplotype of a patient can be determined to provide expected effectiveness of the cancer therapy prior to administering the cancer therapy to the patient. While any suitable methods to accurately map multiple single nucleotide variances in allele-specific manner are contemplated, a preferred method uses phasing of a plurality of RNA molecules in different loci transcribed from a single gene by analyzing the allele fraction of the loci. Most typically, the loci are the non-overlapping portions of the genes, within which at least one allele-specific single nucleotide variance is located. Thus, each RNA molecule transcribed from one locus of the gene contains distinct allele-specific single nucleotide variance (or a set) than another RNA molecule transcribed from another locus of the gene. Preferably, two loci are apart from each other at least 100 base pairs, at least 300 base pairs, at least 500 base pairs, at least 1000 base pairs, or at least 2000 base pairs. Preferably, omics data of each locus of the RNA molecule is obtained through next generation sequencing (RNA-seq) such that the average read length is between 50-500 base pairs, preferably 50-300 base pairs, more preferably between 50-200 base pairs.

In a preferred embodiment, the sequencing depth of each locus is at least 10×, preferably at least 15×, more preferably at least 20×, and most preferably at least 30×. In other words, each single nucleotide variance in each locus in the germline alleles (either maternal or paternal allele) will be covered by at least 10 reads, at least 15 reads, at least 20 reads, or at least 30 reads. The inventors contemplate that the alleles are homozygous where there is only one allele with the requisite read support (all reads correspond to same nucleic acid sequences), and that the alleles are heterozygous where there are two alleles with the requisite read support. Thus, where the alleles are heterozygous, the reads for each locus (10 reads, 20 reads, 30 reads, etc.) can be divided into two groups (e.g., five reads correspond to sequence A and another five reads correspond to sequence B). Thus, for each locus, allele fraction can be calculated based on the ratio of number of reads corresponding to each allele (identified by differential sequences). For example, where the number of reads corresponding to one allele having a single nucleotide variance is 6 out of 20, and the number of reads corresponding to another allele having no single nucleotide variance is 14 out of 20 for the same locus, the allele fraction for the allele having a single nucleotide variance is 0.3 (out of total 1) and the allele fraction for the allele having no single nucleotide variance is 0.7.

Without wishing to be bound by any specific theory, the inventors contemplate that the number of reads by RNA-seq for heterozygous alleles are often imbalanced and such imbalance persists among a plurality of loci of the RNA molecule transcribed from a single gene. Viewed from different perspective, in a single gene, RNA transcripts from each allele are expressed in a specific pattern (e.g., paternal to maternal ratio is 7:3, etc.). Thus, it is likely that a fraction of reads from locus C and a fraction of read from locus D of the RNA transcripts are from the same allele if the fraction ratio to all or another sequence reads of the same locus are same or substantially similar, and as such, a haplotype of locus can be reconstructed based on the allele fraction pattern. For example, the allele fraction of reads having T201 (sequence T in the base pair position 201) is 0.3, the allele fraction of reads having C201 (sequence C in the base pair position 201) is 0.7, the allele fraction of reads having A607 (sequence A in the base pair position 607) is 0.3, and the allele fraction of reads having C607 (sequence C in the base pair position 607) is 0.7. In such case, based on the allele fraction pattern similarity, it can be determined that T201 and A607 are positioned in the same allele while C201 and C607 are positioned in the same allele.

Preferably, the allele fraction that is used to reconstruct the haplotype of the gene is far enough from 0.5 such that two sequences from different alleles are not falsely reconstructed into a single allele or any sequence error in the reads lead to reconstruction of haplotype of two loci from two different allele into a single allele. Thus, the allele fraction is preferably is less than 0.45, preferably less than 0.4, more preferably less than 0.35, or more than 0.55, preferably more than 0.6, or more preferably more than 0.65. In other embodiments, the allele fraction between two alleles differ more than 5%, preferably more than 10%, more preferably more than 20%, or more than 30%.

The types and numbers of genes for allele fraction analysis and reconstruction of haplotype may vary depending on the type of diseases, prognosis of the diseases, and/or desired information (e.g., drug toxicity, drug effectiveness, etc.). For example, where the drug toxicity and/or drug effectiveness in relation to genomic variance is studied, the gene of interest may include genes encoding enzymes that metabolize the cancer drugs in the patient's body, which may include, but not limited to, CYP3A5, CYP2C19, CYP2D6, TPMT, F5, DPYD, G6PD, and NUDT15. Table 2 presents measured frequency of specific allele types among patients using DNA sequencing data analysis as described above. In this study, the inventors developed a clinical pharmacogenomics panel that includes 32 markers (single nucleotide variance) in 10 genes linked to the toxicity of 15 cancer therapies including CYP3A5, CYP2D6, TPMT, F5, DPYD, G6PD, and NUDT15. Tests to determine the haplotypes and presence of marker single nucleotide variance in the haplotype were performed with 1879 patient samples having various types of cancer (e.g., adrenal cancer, bladder cancer, etc.). As shown, the measured frequency is substantially similar to known population frequency (as reported in ExAC database) of the same allele type of the gene. All tests were validated on a cohort of patients previously genotyped by an independent CLIA-validated PCR-based panel, as well as on a set of synthetic data.

TABLE 2

| Gene | Allele | Frequency | Population Frequency |
|---|---|---|---|
| CYP3A5 | *3 | 85.74% | 85-95% |
| CYP3A5 | *6 | 0.43% | 1.19% |
| CYP2D6 | *10 | 4.23% | [2.5-42.4]% |
| TPMT | *3A | 5.69% | 4.50% |
| TPMT | *3B | 5.53% | 2.75% |
| TPMT | *3C | 7.08% | 3.67% |
| TPMT | *2 | 0.16% | 0.14% |
| F5 | rs6025 | 2.13% | 2.15% |
| DPYD | *2A | 0.48% | 0.58% |
| DPYD | rs67376798 | 0.48% | 0.29% |
| G6PD | Mediterranean | 1.22% | 0.24% |
| G6PD | A- | 1.12% | 1.13% |
| NUDT15 | *3 | 1.44% | 2.62% |
| NUDT15 | *4 | 0.08% | 0.24% |

The inventors further studied the prevalence of genomic variance that may affect the cancer drug efficacy or toxicity among patients with various types of cancers. As shown in Table 3, almost all (over 96%) patients having various types of cancers possess at least one genomic variance in at least one gene in the test panel. Furthermore, almost 8% of the patients possess genomic variants that could have resulted life-threatening or severe drug toxicities.

| | | # With | # With Potentially |
|---|---|---|---|
| | | At Least | Treatment-Altering |
| Cancer Type | # Patients | One Variant | Variant(s) (%) |
| Adrenal | 13 | 13 | 2 |
| Bladder | 30 | 30 | 3 |
| Brain | 93 | 91 | 7 |
| Breast | 336 | 317 | 22 |
| Cervical | 16 | 16 | 2 |
| GI Tract | 573 | 556 | 41 |
| Kidney | 38 | 37 | 4 |
| Leukemia | 4 | 4 | 0 |
| Lung | 149 | 143 | 14 |
| Lymphoma | 12 | 12 | 1 |
| Melanoma | 37 | 36 | 1 |
| Mesothelioma | 8 | 8 | 3 |
| Other Cancer | 153 | 148 | 6 |
| Ovarian | 103 | 102 | 8 |
| Prostate | 51 | 49 | 3 |
| Renal Pelvis and Ureter | 10 | 9 | 0 |
| Sarcomas (including | 161 | 154 | 17 |
| Bone) | | | |
| Skin (Non-Melanoma) | 9 | 9 | 1 |
| Testicular | 6 | 6 | 1 |
| Thymic | 17 | 17 | 1 |
| Unknown Primary | 29 | 28 | 1 |
| Uterine (Endometrial) | 29 | 27 | 1 |
| Vulvar | 2 | 2 | 0 |
| Total | 1879 | 1814 | 139 |
| Percent | | 96.54% | 7.40% |

In some embodiments, haplotype determination using RNA phasing can be performed with omics data of the patient's matched normal or healthy tissue and also with omics data obtained of the patient's tumor tissue to determine potentially differential effect and/or toxicity of the cancer therapy. For example, where the healthy tissue and tumor tissue's genomic variances of a gene related to drug toxicity and efficacy are different, systemic drug treatment to the patient may result in severe toxicity only to the healthy tissue and reduced efficacy of drug treatment to the tumor.

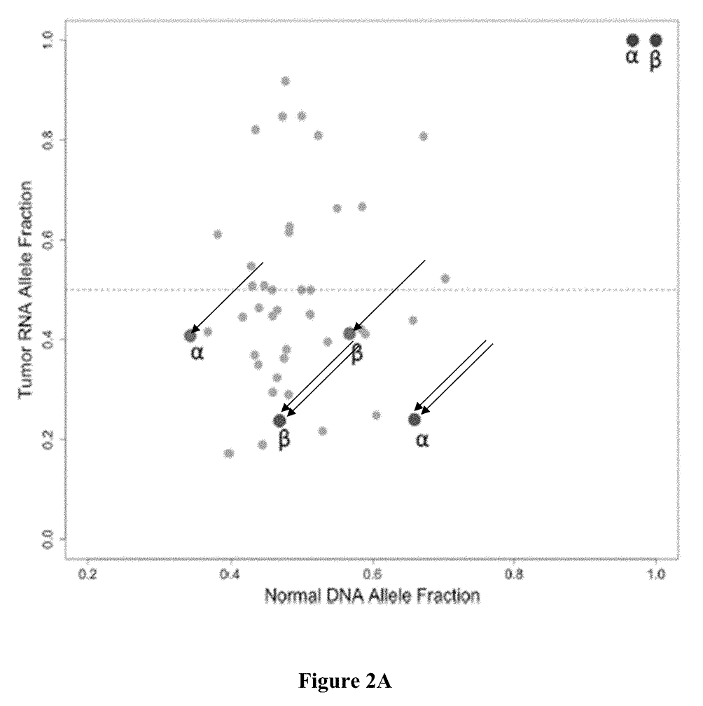

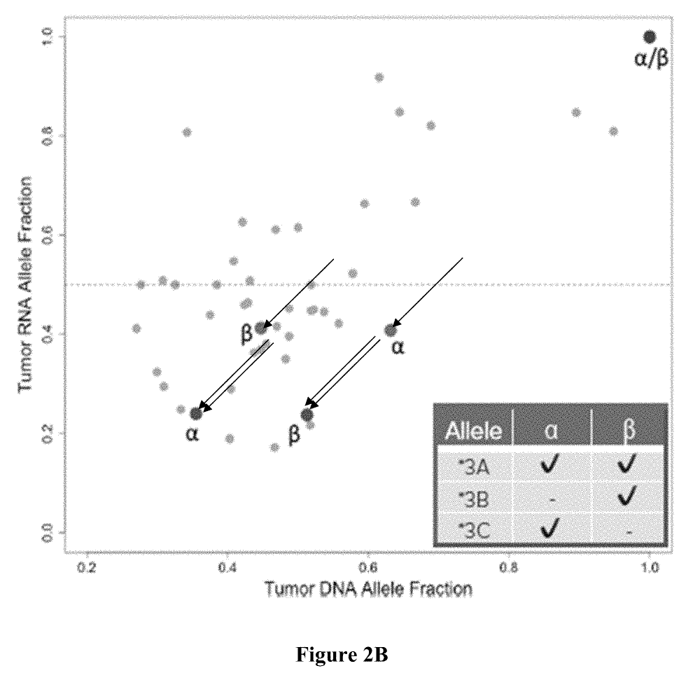

FIGS. 2A and 2B show exemplary allele fraction plot from which the haplotype having two distinct single nucleotide variances in the same allele. In this example, allele fractions of two loci of a tumor RNA transcript having one of single nucleotide variances of TPMT gene are plotted against either normal DNA allele fraction (FIG. 2A) or tumor DNA allele fraction (FIG. 2B). TMPT*3A allele comprises two single nucleotide variances (rs1142345 and rs1800460), each of which are also separately identified as *3B (rs1800460) or as *3C (rs1142345), respectively. If two single nucleotide variances are located in the same allele, the genotype can be identified as *1/*3A. If two single nucleotide variances are located in the different alleles, the genotype can be identified as *3B/*3C. As those two single nucleotide variances are located distantly either in the genome or in the RNA transcript, it is technically impossible to locate two single nucleotide variances via direct phasing using read pairs. The inventors found that at least two patients having two single nucleotide variances (rs1142345 and rs1800460) in the same allele, thus having *1/*3A genotype by determining that allele fractions of two single nucleotide variances are same or substantially similar (e.g., less than 10%, less than 15%, etc.). For example, in the first patient, allele fraction of the first loci of RNA transcript including rs1142345 (shown as a, single arrow) and allele fraction of the second loci of the RNA transcript including rs1800460 (shown as (3, single arrow) are both about 0.4. In another example, in the second patient, allele fraction of the first locus of RNA transcript including rs1142345 (shown as a, double arrow) and allele fraction of the second locus of RNA transcript including rs1800460 (shown as (3, double arrow) are both about 0.2.

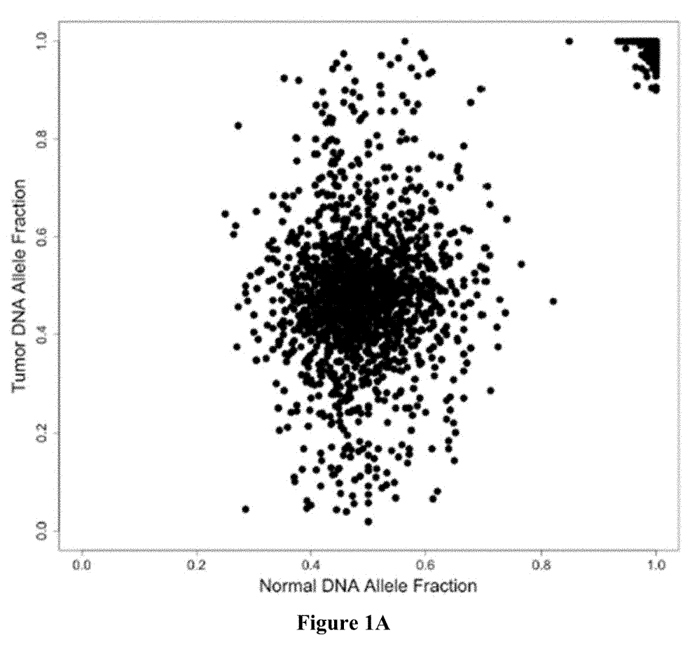

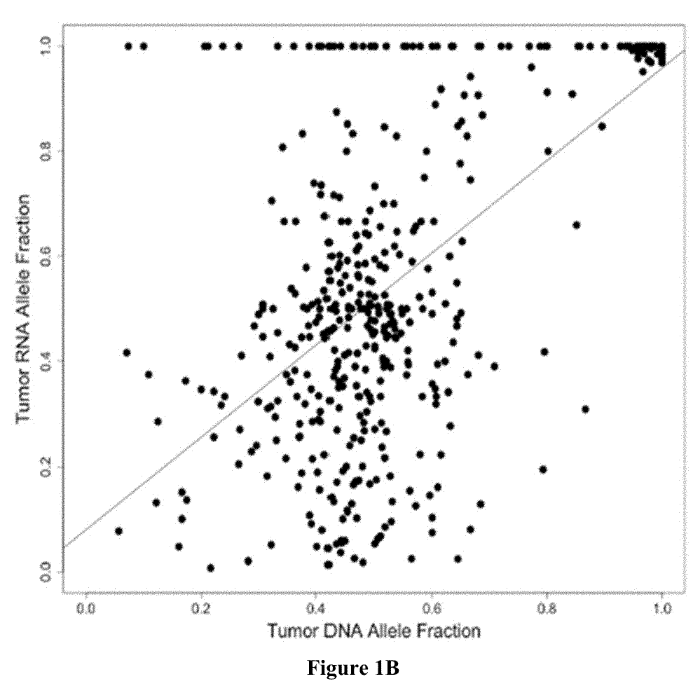

In another example, where tumor tissue possess further genomic variance due to the allele specific deletions and/or amplifications, tumor tissue may have different sensitivity or tolerance to the toxicity of the cancer therapy due to reduced or enhanced phenotype from the deleted or amplified haplotype relative to the intact haplotype. As shown in FIG. 1A, DNA allele fractions in healthy tissue, in majority, between 0.4 and 0.6, indicate that the copy numbers of two alleles of a given gene is substantially homogenous and that few allele-specific amplification or deletion events are present in the healthy tissue genome. In contrast, DNA allele fractions in tumor tissue are more widely distributed between 0 and 1, indicating that there are substantial imbalances between copy numbers of two alleles in substantial number of genes in the tumor cells, potentially due to the allele-specific amplification or deletion events.

FIG. 1B shows correlations of DNA allele fraction and RNA allele fraction for a plurality of loci in the genes in the tumor tissue. As shown, RNA allele fractions of many genes are distinct from its corresponding DNA allele fractions, indicating that at least two factors: allele-specific DNA copy number (e.g., by allele-specific amplification or deletion) and imbalance of allele-specific transcription levels of a gene transcript, may affect tumor-specific drug sensitivity and/or toxicity compared to healthy tissue in the same patients.

Thus, in some embodiments, the inventors contemplate that genomics data analysis on the genes linked to the toxicity of cancer therapies (e.g., CYP3A5, CYP2C19, CYP2D6, TPMT, F5, DPYD, G6PD, and NUDT15) with respect to deletion or amplification of allele(s). Deletion or amplification of an allele of a gene can be determined by counting allele-specific copy number of specific genomic regions. Most typically, allele specific copy number is calculated using a dynamic windowing approach that expands and contracts the windows genomic width according to the coverage in either the tumor or normal germline data of the genes having or expected to have heterozygous alleles. The process is initialized with a window of zero width. Each unique read from either the tumor or germline sequence data will be tallied into tumor counts, Nt, or germline counts, Ng. The start and stop positions of each read will define the window's region, expanding as new reads exceed the boundaries of the current window. When either the tumor or germline counts exceed a user-defined threshold, the window's size and location are recorded, as well as the Nt, Ng, and relative coverage Nt. Tailoring the size of the Ng window according to the local read coverage will create large windows in regions of low coverage (for example, repetitive regions) or small windows in regions exhibiting somatic amplification, thereby increasing the genomic resolution of amplicons and increasing our ability to define the boundaries of the amplification. More detailed procedure is described in U.S. Pat. No. 9,824,181, which is incorporated by reference.

It is contemplated that allele-specific copy number is used to identify genomic regions exhibiting loss-of-heterozygosity (both copy-neutral and copy-loss) as well as amplifications or deletions specific to a single allele. This last point is especially important to help distinguish potentially disease-causing alleles as those that are either amplified or not-deleted in the tumor sequence data. Furthermore, regions that experience hemizygous loss (for example, one parental chromosome arm) can be used to directly estimate the amount of normal contaminant in the sequenced tumor sample.

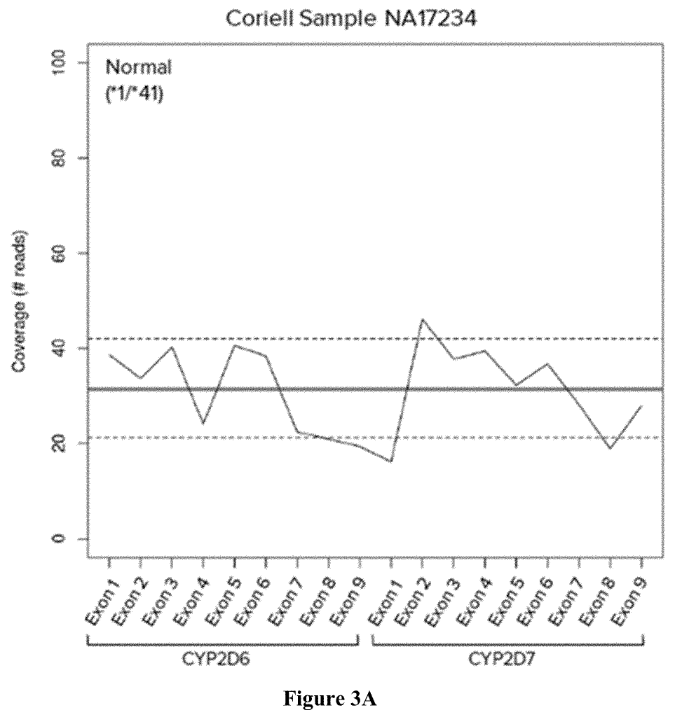

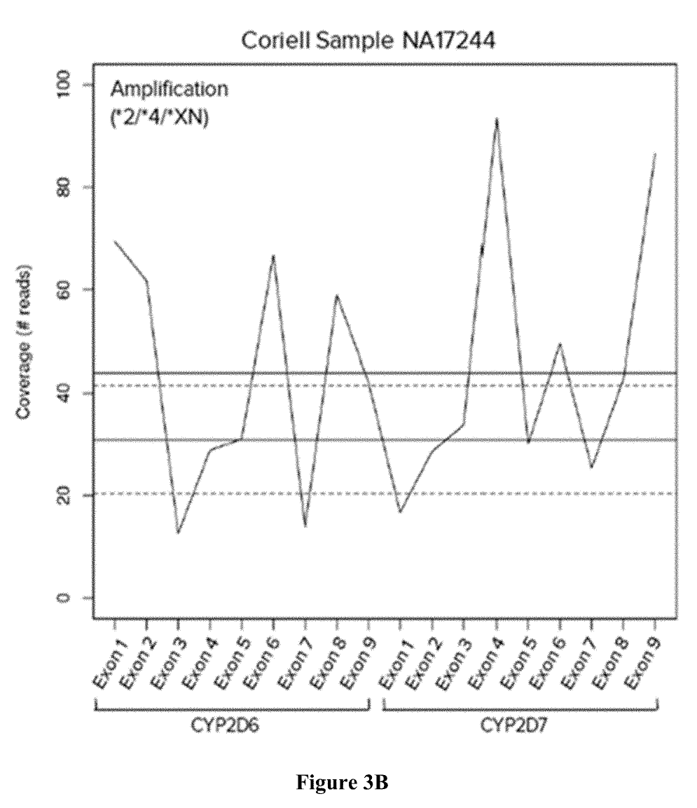

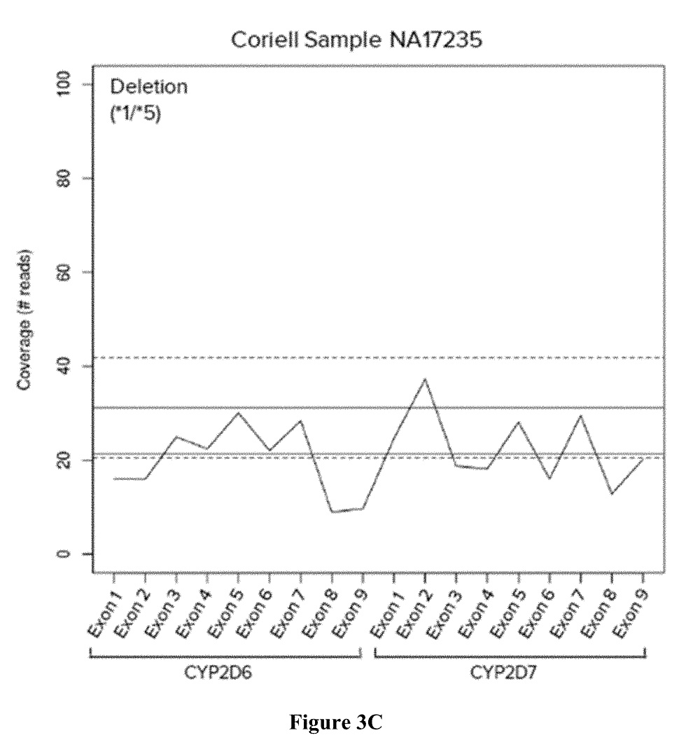

FIGS. 3A-C show exemplary graphs of copy numbers (shown as read coverage, or read numbers) of individual exons of CYP2D6 (exons 1-9) and CYP2D7 (exons 1-9). As shown, in sample NA17234 that has normal allele genotypes (*1/*41) without deletion or amplification in exons of CYP2D6 and CYP2D7), the average number of copy numbers is about 30 with a standard deviation of ±10 (FIG. 3A). In contrast, in sample NA17244, the average number of copy numbers is increased to over 40, indicating that there are amplifications in some of exons in either CYP2D6 and CYP2D7 (FIG. 3B). Specifically, for example, exon 6, exon 8 of CYP2D6, and exon 4 and exon 9 of CYP2D7 show copy numbers that are increased 50-100% compared to copy numbers of sample NA17244, indicating that one of the alleles of those exons may be amplified. In addition, in sample NA17235, the average number of copy numbers is decreased to about 20, indicating that there may be deletions in some of exons in either CYP2D6 and CYP2D7 (FIG. 3C). Specifically, for example, exon 1 and exon 2 of CYP2D6 show copy numbers that are decreased to around half of the normal genotype (NA17244), indicating that one of the alleles of those exons may be deleted.

Such obtained RNA phasing information and genomic copy number information can be taken together to identify differential allele haplotypes in tumor and/or healthy tissues. For example, for each healthy and tumor tissue, allele haplotype in relation to a plurality of single nucleotide variances can be identified and determined using RNA phasing as described above. In addition, by analyzing whole genome copy number or exome copy number for each exon, allele haplotype in relation to amplification and/or deletion in one or more of a portion of exons.

The inventors further contemplate that such identified allele haplotypes can be associated with effectiveness and/or toxicity of specific drug in specific cancer. For example, CYP2D6 enzyme catalyzes the metabolism of a large number of clinically important drugs including cancer drugs and opioids. Various alleles having different combinations of single nucleotide variances and/or deletions have been identified in relation to the activity of the CYP2D6 enzyme (e.g., normal function, decreased function, no function, etc.). It is expected that where the CYP2D6 gene include a haplotype that causes decreased function or no function of the CYP2D6 enzyme, the cancer drug or therapy may have increased toxicity to the tissue as the cancer drug is likely to be catalyzed more slowly. Such increased toxicity by the cancer drug could render a harmful effect to the healthy tissue, especially to the liver tissue, where the systemically circulating drugs are metabolized. Conversely, it is expected that where the CYP2D6 gene include a haplotype that causes increased function of the CYP2D6 enzyme, for example, due to the amplification of genes and number of normal function enzymes produced, the cancer drug or therapy may have decreased effectiveness as the cancer drug is likely to be catalyzed too quickly.

In addition, the inventors also contemplate that the effectiveness and/or toxicity of specific drug in specific cancer can be assessed by comparing and/or analyzing the allele haplotypes of tumor tissue and the healthy tissue of the patient. For example, a tumor tissue may have a gene with different haplotype(s) (e.g., different combinations of single nucleotide variances and/or amplification or deletion of exons, etc.) from that of healthy tissue, which may result in differential response to the drug or differential toxicity from the exposure to the drug.

Consequently, the overall effectiveness and/or toxicity of a cancer drug or therapy to treat specific type of cancer of the patient can be estimated, calculated and/or inferred from the determined allele haplotype and the combination of allele haplotypes of the gene of the patient. Most typically, from the pathway analysis of the patient omics data, couple of cancer treatment or cancer drug can be selected that are likely to have positive outcome to treat the cancer of the patient. Then, based on the selected cancer treatment and/or drug, one or more genes that are related to the sensitivity, effectiveness, and/or toxicity to or by the selected cancer treatment and/or drug can be chosen for haplotype analysis. Haplotype analysis using RNA phasing and genomic copy number analysis can determine haplotype of each allele of the selected genes, and each haplotype of each allele can be assigned or provided with a quantifiable score or value with respect to the sensitivity, effectiveness, and/or toxicity to or by the selected cancer treatment and/or drug. For example, where CYP2D6 gene of the patient have two alleles: one associated with decreased enzyme function and another associated with normal enzyme function, the allele associated with decreased enzyme function can be scored with lesser valued score than the allele associated with normal enzyme function. Additionally, where the allele associated with normal enzyme function is amplified, then such allele can be assigned with even higher score than the allele associated with decreased enzyme function. Scores from each allele can be combined or taken together to calculate the overall score for the gene with respect to the sensitivity, effectiveness, and/or toxicity to or by the selected cancer treatment and/or drug. Thus, it should be appreciated that the score assigned for haplotype of the allele may differ for the same gene depending on the types of response (sensitivity, effectiveness, and/or toxicity), types of cancer treatment and/or drug, and/or types of cancer.

In some embodiments, the scores calculated from alleles of genes in the healthy tissue and tumor tissue can be compared to calculate an optimum score of the gene to the treatment. For example, where the alleles of the gene in the healthy tissue is associated with high risk of toxicity while the alleles of the gene in the tumor tissue is associated with the low effectiveness of the cancer drug, then the optimum score for the gene to the cancer drug will be low as a combination (e.g., sum of two scores) of low score (or even negative score) for high toxicity to the healthy tissue and the low score for low effectiveness to the tumor tissue.

The inventors further contemplate that, based on the allele haplotype information, especially the score of each allele of the gene, the score of the gene having heterogeneous alleles, or the optimum score for the gene in association with the cancer drug effectiveness and/or toxicity, a patient's record can be generated or updated, a new treatment plan can be recommended, or a previously used treatment plan can be updated. For example, where the optimum score for the gene in association with the cancer drug effectiveness and/or toxicity is low, indicating possible high toxicity to the healthy tissue without desirable amount of effect to the tumor tissue, the patient's record can be updated with the allele information and/or score calculated based on the allele information, and optionally with a recommendation not to use such treatment or cancer drug to the patient, with or without an expected outcome and side effects in order to avoid potential adverse effect of such treatment or cancer drug to the patient.

In some embodiments, based on the allele haplotype information, especially the score of each allele of the gene, the score of the gene having heterogeneous alleles, or the optimum score for the gene in association with the cancer drug effectiveness and/or toxicity, the treatment regimen to the patient can be adjusted or modified. For example, where the optimum score for the gene in association with the cancer drug effectiveness and/or toxicity is medium, indicating a likelihood of success in treating the tumor cell with the cancer drug, yet possible high toxicity to the healthy tissue, a dose and/or a schedule of administering the cancer drug can be changed (e.g., smaller dose to so reduce the toxicity to the healthy tissue and/or less frequency in administering the drug (e.g., once a day instead of twice a day, etc.), more frequent administration schedule with the same dose of drug to overcome the fast metabolism of the drug, etc.).

Alternatively and/or additionally, the method of treatment for the same cancer drug can be changed based on the allele haplotype information, especially the score of each allele of the gene, the score of the gene having heterogeneous alleles, or the optimum score for the gene in association with the cancer drug effectiveness and/or toxicity. For example, where the optimum score for the gene in association with the cancer drug effectiveness and/or toxicity is medium, indicating a likelihood of success in treating the tumor cell with the cancer drug, yet possible high toxicity to the healthy tissue, it may be recommended that the method of administering the cancer drug to the patient can be changed from systemic administration (e.g., intravenous injection, etc.) to local administration (e.g., intratumoral injection) in order to minimize the exposure of the healthy tissue to the cancer drug before the cancer drug reaches to the tumor.

The inventors have further disclosed herein an integrative panomic approach to pharmacogenomics screening. The screening test disclosed herein screens for pharmacogenomics variants related to 19 gene-drug pairs with CPIC guidelines and FDA label indications. The inventors performed pharmacogenomics screening on whole genome and whole exome sequencing data of FFPE tumors and matched normals from 1,879 oncology patients. Patients were screened using a panel of 31 germline markers in 11 genes linked to toxicities from 14 cancer therapies, as shown in Table 4. The test has been validated on 10 cell lines from the CDC GeT-RM, on a set of synthetic data, as well as on a cohort of patients previously genotyped by an independent CLIA-validated PCR-based panel. The inventors found that, of the 1879 patients screened, 96.4% contained a variant with a pharmacogenomics recommendation. Furthermore, 6.8% of patients had genomic variants associated with severe or life-threatening drug toxicities. For all alleles in the inventors' clinical panel, similar allele frequencies to those reported in the ExAC database were observed. In all validation studies, the inventors demonstrated that the test detects each variant in the panel, and correctly determines patient genotype in all studied cases.

TABLE 4

| Drugs | Gene | Clinical Implications |
|---|---|---|
| Tamoxifen | CYP2D6 | Lower endoxifen concentration, increased |
| | | likelihood of recurrence |
| Lapatinib | HLA-DRB1 | Increased risk of hepatotoxicity |
| | HLA-DQA1 | |
| Fluorouracil, Capecitabine, | DPYD | Increased risk of severe or life threatening |
| Tegafur | | adverse events |
| Belinostat, Irinotecan, Nilotinib, | UGT1A1 | Increased risk of toxicities, neutropenia, |
| Pazopanib | | hyperbilirubinemia, hyperbilirubinemia |
| | | (respectively) |
| Rasburicase, Dabrafenib | G6PD | Increased risk of hemolytic anemia |
| Tacrolimus | CYP3A5 | Normal metabolizers may fail to reach target dose |
| Mercaptopurine | NUDT15 | Increased risk of myelotoxicity (leukopenia or |
| | | neutropenia) |
| Azathioprine, Mercaptopurine, | TPMT | Increased risk of myelosupression and potentially |
| Thioguanine | | fatal toxicities |
| Eltrombopag Olamine | F5 | Increased risk of thromboembolism |

Thus, the screening test disclosed herein was able to accurately detect pharmacogenomic variants in oncology patients. Observed allele frequencies correspond well to known population frequencies, and validation studies demonstrated that the test detects each variant in our panel, and correctly determines patient genotype in all studied cases. Given the high percentage of patients with potentially treatment-altering genomic variants, these results underscore the need for more routine pharmacogenomics screening in the oncological setting.

In the inventors' study, most patients (>96%) had at least one variant screened for in the pharmacogenomics panel covering 16 commonly used drugs. Of those, a surprising percentage had variants with the potential to change treatment due to severe or life-threatening implications. In a cohort of patients with HR+ breast cancer, large percentage had the CYP2D6*10 haplotype, indicating potential toxicities when treated with Tamoxifen. Thus, in one embodiment, these results underscore the need for pharmacogenomics screening for all patients undergoing cancer treatment.

It should be appreciated that the inventive subject matter uses comprehensive pathway analysis using various types of omics data to identify the cancer treatment or cancer drugs having high likelihood of success in treating the tumor. Further, the inventive subject matter uses comprehensive analysis on allele haplotype(s) of heterogeneous alleles carrying allele-specific single nucleotide variances and/or amplifications/deletions using RNA-seq phasing and DNA copy number analysis to predict effectiveness and/or toxicity of a cancer treatment in a patient-specific manner. Thus, this approach allows streamlined customization of cancer treatment regimen to maximize the effectiveness while avoiding any adverse effects of the cancer treatment, including possible life-threatening side effect.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of determining effectiveness of a cancer therapy in a patient having a tumor, comprising:
obtaining whole genome or whole exome sequencing data from tumor sample and matched normal sample of the patient, wherein the whole genome or whole exome sequencing data comprises allele fraction information of two or more RNA loci of an RNA molecule transcribed from the gene, and wherein the two or more RNA loci have two or more nucleotide variations;
using allele fraction information to reconstruct a haplotype of the tumor sample and matched normal sample;
predicting whether a gene product has normal function, reduced function, or no function based on the allele fraction information and haplotype of the tumor sample; and
determining the effectiveness of the cancer therapy based on the predicted function of the gene product.

2. The method of claim 1, wherein the gene is at least one of CYP3A5, CYP2D6, TPMT, F5, DPYD, G6PD, and NUDT15.

3. The method of claim 1, wherein the gene is at least one of HLA-DRB1, HLA-DQA1, and UGT1A1.

4. The method of claim 1, the haplotype is reconstructed to have the two or more nucleotide variations in an allele of the gene when the allele fractions of the loci having the two or more nucleotide variations differ less than 10%.

5. The method of claim 1, wherein the whole genome or whole exome sequencing data comprises a copy number of the two or more loci, and further comprising:
determining amplification of at least one of the two or more RNA loci;
generating or updating the patient's record with amplification information of the gene in relation to the expected effectiveness of a cancer therapy.

6. The method of claim 1, further comprising adjusting recommended dose and schedule of the cancer therapy based on the expected effectiveness.

7. The method of claim 1, wherein the whole genome or whole exome sequencing data further comprises allele fraction information of two or more RNA loci.

8. The method of claim 1, further comprising:
using the allele fraction information derived from the healthy tissue to reconstruct a healthy tissue haplotype;
comparing the allele fraction information derived from the tumor tissue with the allele fraction information derived from the healthy tissue to obtain tumor-specific allele fraction information; and
generating or updating the patient's record with the allele fraction information and the tumor-specific allele fraction information.

9. The method of claim 8, further comprising adjusting recommended dose and schedule of the cancer therapy based on a comparison of the reconstructed healthy tissue's haplotype and the tumor-specific haplotype.

10. A method of treating a patient having a tumor, comprising:
obtaining the patient's whole genome or whole exome sequencing data comprising allele fraction information of two or more RNA loci of an RNA molecule transcribed from a gene, wherein the two or more RNA loci have two or more nucleotide variations, respectively;
using allele fraction information to reconstruct a haplotype of the two or more RNA loci;
inferring an expected effectiveness of a cancer therapy for the haplotype; and
treating the patient by adjusting recommended dose and schedule of the cancer therapy based on the expected effectiveness.

11. The method of claim 10, wherein the allele fraction information of the two or more RNA loci is derived from the tumor of the patient.

12. The method of claim 10, wherein the gene is at least one of CYP3A5, CYP2D6, TPMT, F5, DPYD, G6PD, and NUDT15.

13. The method of claim 1, wherein the gene is at least one of HLA-DRB1, HLA-DQA1, and UGT1A1.

14. The method of claim 10, wherein the two or more RNA loci are at least 300 bp apart.

15. The method of claim 10, wherein the whole genome or whole exome sequencing data comprises a copy number of the two or more loci, and further comprising:
determining amplification of at least one of two or more loci;
adjusting recommended dose and schedule of the cancer therapy with amplification information of the gene in relation to the expected effectiveness of a cancer therapy.

16. The method of claim 15, wherein the whole genome or whole exome sequencing data further comprises allele fraction information of the two or more RNA loci derived from a healthy tissue of the patient.

17. The method of claim 16, further comprising:
using the allele fraction information derived from the healthy tissue to reconstruct a healthy tissue haplotype;
comparing the allele fraction information derived from the tumor tissue with the allele fraction information derived from the healthy tissue to obtain tumor-specific allele fraction information; and
adjusting recommended dose and schedule of the cancer therapy based on a comparison of the reconstructed healthy tissue's haplotype and the tumor-specific haplotype.

18. The method of claim 17, further comprising generating or updating the patient's record with the allele fraction information and the tumor-specific allele fraction information.

19. The method of claim 11, wherein the cancer therapy is identified by a pathway analysis using at least two of genomics, transcriptomics, and proteomics data of the patient.
